(19) United States
(12) Patent Application Publication
(10) Pub. No.: US 2005/0124594 A1
(43) Pub. Date: Jun. 9, 2005

(54) Method of treatment

(76) Inventors: Mark Cedric Gillies, Sydney (AU); Philip Leslie Penfold, Sydney (AU); Francis Alfred Billson, Sydney (AU)

Correspondence Address: FROMMER LAWRENCE & HAUG, 745 FIFTH AVENUE- 10TH FL., NEW YORK, NY, 10151, US

(21) Appl. No.: 10/966,627

(22) Filed: Oct. 15, 2004

(30) Foreign Application Priority Data
Jul. 10, 1998 (AU) PP4607
Sep. 11, 1998 (AU) PP 5847

(51) Int. Cl. A61K 31/58

(52) U.S. Cl. 514/174

(57) ABSTRACT

This invention relates to the prophylaxis of choroidal neovascularisation in macular degeneration by the introduction of a suitable anti-inflammatory agent into the vitreous. In particular, it relates to the prophylaxis of neovascularisation with an anti-inflammatory steroid, such as an 11-substituted 16 alpha, 17 alpha-substituted methylenedioxy steroid of formula (I) wherein (a) is (b), (c), (d), (e), (f), (g), (h), (i), (j), (k) or (l); R1 and R2 are hydrogen or alkyl; -Ca-Cb- is —CH2—CH2—, —CH═CH—, (m) or (n); R3 is methyl, hydroxymethyl or alkylcarbonyloxymethyl, methylaminoalkylenecarbonyloxymethyl, or phenylaminoalkylenecarbonyloxymethyl; R4 is alkanoyl; and X is halogen in eyes which have been identified as having a high risk of developing choroidal neovascularisation. More particularly, it relates to prophylaxis with triamcinolone acetonide, (compound II).

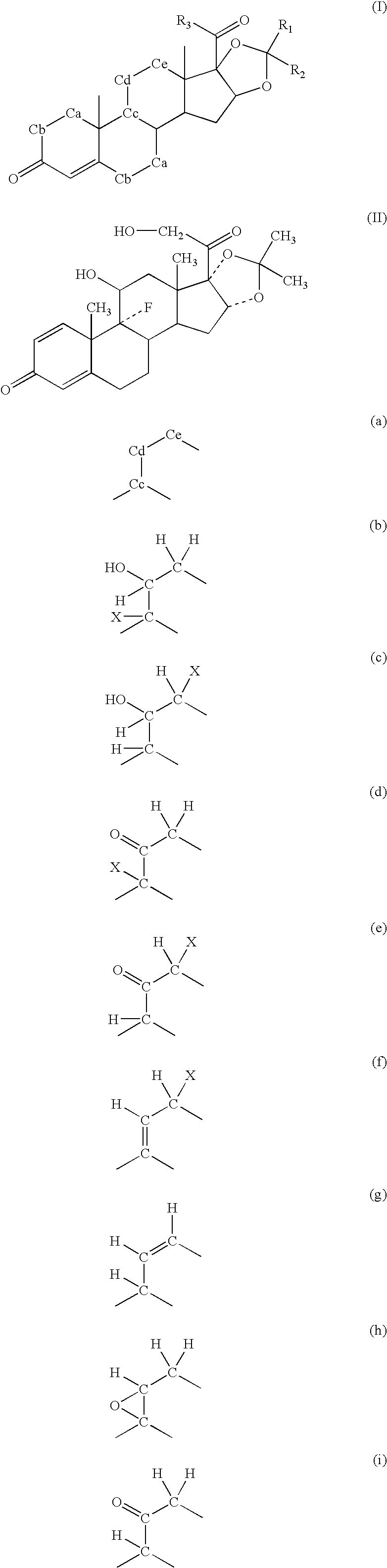

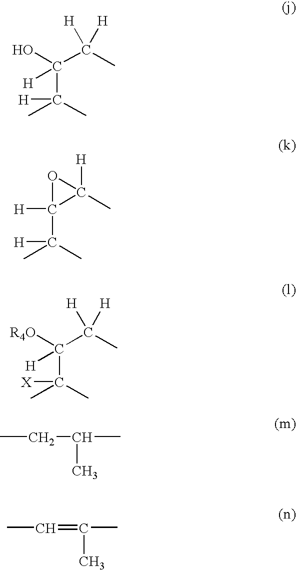

### FIELD OF THE INVENTION

This invention relates to the prophylaxis of choroidal neovascularisation in macular degeneration by the introduction of a suitable anti-inflammatory agent into the vitreous. In particular, it relates to the prophylaxis of neovascularisation with an anti-inflammatory steroid in eyes which have been identified as having a high risk of developing choroidal neovascularisation. More particularly, it relates to prophylaxis with triamcinolone acetonide.

### BACKGROUND OF THE INVENTION

Choroidal neovascularisation (CNV) is the commonest cause of severe visual loss in age related macular degeneration (ARMD). ARMD is itself the commonest cause of blindness in the developed world. The Blue Mountains Eye Study found that 1.2% of the population 43 or older had active CNV, increasing to 19.6% of those 85 or older. These results are very similar to those found by studies in the U.S.A. and Europe (Beaver Dam and Rotterdam studies). Of the seventeen people regarded as legally blind in the Blue Mountains study, 15 (88%) suffered ARMD as their principal ophthalmic disease. The last review of blindness registrations in Australia examined the data in Western Australia from 1984 to 1988. There were more registrations due to ARMD each year than due to all other causes put together. With life expectancy increasing by the year, exudative ARMD is becoming a major epidemic.

Current treatment of established CNV is generally unsatisfactory. Only around 15% of cases of CNV in ARMD (or “Exudative ARMD”) can be ablated with a laser without loss of central vision and at least one half of these eyes suffer recurrences or develop new CNV within five years (MPS91, MPS94). The efficacy of other forms of therapy, such as surgical excision and teletherapy has not yet been established. Preliminary results of these treatments are not, however, particularly encouraging.

Bearing in mind that most patients are only really blinded when both eyes are affected, effective prophylactic treatment of the second eye when a patient presents with loss of vision in the first eye may be the most practical way to reduce the prevalence of blindness from ARMD. Up to 87% of patients with age-related macular degeneration who develop choroidal neovascularisation in one eye will develop the same problem in the other eye within five years.

Most of the pathological studies published to date have concentrated on the associations of ARMD, such as drusen, which, whilst of undoubted value, suggest few potential interventions. On the other hand, current understanding of angiogenesis suggests that CNV in ARMD arises and is governed in response to external influences which may, in turn, be susceptible to pharmacological modulation.

One influence which is potentially treatable is the inflammation which a substantial body of evidence has linked with the pathogenesis of ARMD. Autoantibodies directed against both neuronal and glial elements of the retina occur early in the course of the disease. Immunocompetent cells are found on microscopic examination of both neovascular and atrophic maculae. While these may be epiphenomena, a critical role for activated immunocompetent cells in CNV is strongly suggested by their prominence in the very earliest through to the late phases of growth of CNV. This is consistent with the release by macrophages of angiogenic factors under hypoxic conditions and the ability of leukocytes to influence angiogenesis, including normal angiogenesis of the human choroidal and retinal vasculature. The origin of these immunocompetent cells may be choroidal and/or microglial cells of the retina itself. The expression of CD45, MHC class II and macrophage antigens by human retinal microglia indicates they have the potential to promote CNV.

Our U.S. patent (U.S. Pat. No. 5,770,589) is directed to the treatment of established CNV in age-related macular degeneration, with an injection into the vitreous humour of an anti-inflammatory steroid, preferably triamcinolone acetonide. U.S. Pat. No. 5,770,589 is thus restricted to persons who suffer age-related macular degeneration where CNV is established.

The disclosure of U.S. Pat. No. 5,770,589 is incorporated herein by reference. This disclosure in general, and examples 3 and 4 in particular provide sufficient evidence from a histopathological and clinical point of view that triamcinolone which has been introduced into the vitreous, modulates the resident immune cell activity which leads to control and resorbtion of exudation and improved visual acuity. The loss of vision in exudative macular degeneration is a direct result of growth and exudation of choroidal vessels into the neural retina which leads to the loss of photo receptor function. The method of treatment described and claimed in this document leads to improved visual acuity and in this respect is both a method of treatment and a method for the prophylaxis of further loss of visual acuity in a patient already suffering from CNV in macular degeneration.

Until the present invention, however, there has been no way of treating prophylactically either a person who does not show any CNV in ARMD or confidently predicting whether CNV would occur in the fellow eye of one which presently demonstrates this condition.

### DISCLOSURE OF THE INVENTION

According to a first aspect of this invention there is provided a method for the prevention of choroidal neovascularisation in macular degeneration in a patient requiring said prevention, comprising introducing into the vitreous of said patient an effective amount of an anti-inflammatory steroid or an ophthalmologically acceptable composition or formulation containing said anti-inflammatory steroid wherein said patient does not have choroidal neovascularisation in the eye to be treated but has an increased risk factor of developing choroidal neovascularisation.

According to a second aspect of this invention there is provided an anti-inflammatory steroid or an ophthalmologically acceptable composition or formulation containing said anti-inflammatory steroid, when used in the prevention of choroidal neovascularisation in macular degeneration, said prevention as broadly described in the first aspect of this invention and more specifically as herein below described.

According to a third aspect of this invention there is provided an anti-inflammatory steroid or an ophthalmologically acceptable composition or formulation containing said anti-inflammatory steroid, for use in the prevention of choroidal neovascularisation in macular degeneration, said prevention as broadly described in the first aspect of this invention and more specifically as herein below described.

According to a fourth aspect of this invention there is provided the use of an anti-inflammatory steroid or an ophthalmologically acceptable composition or formulation containing said anti-inflammatory steroid, for the manufacture of a medicament for the prevention of choroidal neovascularisation in macular degeneration, said prevention as broadly described in the first aspect of this invention and more specifically as herein below described.

The use of an anti-inflammatory steroid or an ophthalmologically acceptable composition or formulation containing said anti-inflammatory steroid, for the manufacture of a medicament for the prevention of choroidal neovascularisation in macular degeneration.

The anti-inflammatory steroid used in this invention is preferably in crystalline form and is more preferably sparingly soluble in the vitreous of the eye.

Preferred steroids include 11-substituted 16α,17α-substituted methylenedioxy steroids of the formula 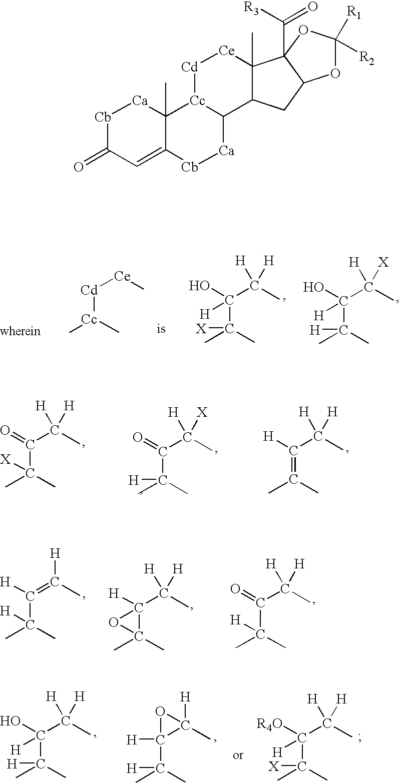
R₁ and R₂ are hydrogen or alkyl; -Cₐ-C_{b}- is —CH₂—CH₂—, —CH═CH—, 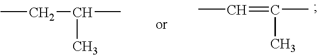
R₃ is methyl, hydroxymethyl or alkylcarbonyloxymethyl, methylaminoalkylenecarbonyloxymethyl, or phenylaminoalkylenecarbonyloxymentyl; R₄ is alkanoyl; and X is halogen.

More preferred are compounds of the formula: 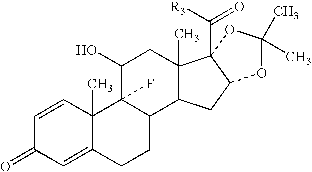
wherein R₃ is hydroxymethyl, phenylcarbonylaminoisopropylcarbonyloxymethyl, or 2,2-dimethylpropylcarbonyloxymethyl.

The preferred steroid is crystalline 9-fluoro-11,21-dihydroxy-16,17-[1-(methylethylidine)bis (oxy)]pregna-1,4-diene-3,20-dione;

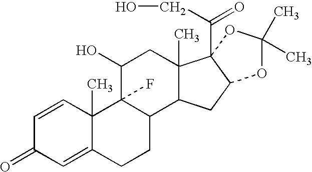

This compound, also known by its generic name as triamcinolone acetonide is suitably prepared by known methods.

Another suitable steroid is 6,9-difluoro-11,21-dihydroxy-16,17-[(1-methylethylidene)bis(oxy)]pregna-1,4-diene-3,20-dione:

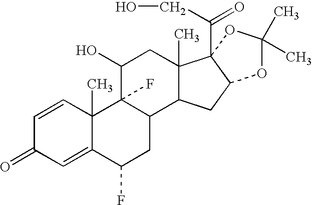

This compound, also known-by its generic name as fluocinolone acetonide is suitably prepared by known methods.

The steroids are preferably crystalline or lipophilic and are administered in distilled water only, or with a minimum of carriers or adjuvants. However, a depot pharmaceutical composition comprising an effective amount of said antiinflammatory steroid together with a pharmaceutically and opthalmologically acceptable carrier, diluent and/or excipient may be used (eg Kenalog).

When triamcinolone acetonide is used, such a preparation may be made up by using Kenacort-A40 (registered trade mark) (Squibb) as the anti-inflammatory steroid. Suitable pharmaceutically acceptable salts of this compound may be used. For example, the acetate of triamcinolone acetonide may be used.

As the steroids suitable for use in this invention are sparingly soluble in the vitreous, crystalline forms are suitable for administration. The steroids may be formulated with carriers, diluents and/or excipients which are compatible with the vitreous and which do not leave any vision impairing residue in the eye.

The compositions of this invention may be administered as above or in slow release devices. The latter are preparations in which the release of a drug is prolonged by a variety of mechanisms. These include: non-erodible devices, for example where a drug is contained within a compartment enveloped by a permeable or semi-permeable membrane or equivalent structure; remote and/or refillable reservoirs. Also included are biodegradable preparations such as biodegradable particles in which the polymer chemistry is manipulated to change the release rate of the drug, for example by using polylactic glycolic acid; biodegradable micro-and nano-particles; liposomes; drug-drug conjugates; or polymer-drug conjugates.

The composition of the present invention is suitably administered by intravitreal injection by methods known in the art. For example, the eye is washed with a sterilising agent such as Betadine and a topical anaesthetic and the steroid is injected in distilled water with a fine gauge (e.g. 30 gauge) needle at a position in the eye such that the steroid crystals will settle to the posterior pole towards the ventral surface. It may also be necessary to prepare the eye for injection by application of positive pressure prior to injection.

The steroid should be as concentrated as feasible to minimise the volume to be injected. The dosage of a single injection of triamcinolone, for example, may be between about 1 mg and about 8 mg. Typically, 4mg of steroid is deposited intravitreally and thus it is necessary to inject 0.1 mL of Kenacort-A40 solution.

In addition, the compositions or devices to deliver these compositions may be introduced into the eye by for example iontophoresis; through an indwelling catheter or similar device such as a tube or an injection port; or through a surgical incision. These manipulations are usually, but not always, performed through the pars plana approach to the posterior segment.

The compositions of this invention may also be presented as a unit dose in a syringe ready for administration.

The method of the present invention may be practised alone or in conjunction with other therapy. Where laser treatment of the retina is attempted in an effort to clear drusen, steroid may be injected before or after the, laser treatment.

Suitably, a patient who is in need of such prophylaxis is one who has an increased risk of developing CNV according to the criteria of either group A or group B as follows:

Group A
There is no evidence of CNV in the eye in need of treatment but there is CNV in the fellow eye.
The patient has any of the following four high risk factors:
   ≧5 drusen which are larger than 65 μm in diameter,
   Focal hyperpigmentation,
   ≧1 large druse,
   Systemic hypertension.

Group B
There is no neovascularisation in either eye, however there are soft drusen, pigment clumps or “pseudodrusen” in either eye.

Additional Criteria

The following criteria also apply to patients in either group A or group B.
The patient either has a family history of CNV or is genetically predisposed to it.
Evidence that the patient has an immune response directed against the retina. For example, patients with inflammatory diseases of the choroid frequently develop CNV. Retinal antibodies may or may not be present in the serum in this condition.
The patient is about to undergo intraocular surgery eg removal of a cataract.

It is recognised that these factors indicate that an eye is at risk. (MPS Group, Arch Ophthmalol, 1997 Vol 115 pp 741-747).

Choroidal neovascularisation includes classic and occult neovascularisation.

Optionally, more than one treatment with anti-inflammatory steroid may be administered. As mentioned earlier, the anti-inflammatory steroid of preference is triamcinolone acetonide. When triamcinolone acetonide is used, the period of time between injections is at least six months. Preferably, the period of time between injections is 12 months. The period for continuing treatment is indefinite.

### BEST AND OTHER MODES FOR CARRYING OUT THE INVENTION

The present invention is further illustrated by way of the following Examples which are not to be construed as, limiting on the scope of the invention thereof.

### EXAMPLE 1

A patient in whom prophylactic treatment was used was an 82 year old female. There was marked macula degeneration in both eyes. She underwent cataract surgery late in July 1995 and developed neovascularisation of the right macula within three weeks. She also had cataract in the left eye but surgery was deferred for fear of developing the same complication. In the left macula there were greater than 5 drusen, some of them larger than 500 μm, and coarse pigment clumping, all high risk features. The cataract in the left eye continued to advance. By August 1997 it was very dense, reducing the visual acuity to 6/24. In spite of the high risk of neovascularisation, she underwent surgery in October 1997. As a prophylactic measure to reduce the risk of subsequent CNV, she received 40 mg of triamcinolone to the orbital floor beneath the eye at the time of surgery. The visual acuity improved to 6/12. She progressed well until June 1998 when she developed left CNV. By this time the effect of the triamcinolone had worn off. It was felt likely that the tramcinolone had delayed the formation of CNV after surgery. Although the triamcinolone was not applied into the eye, but around it, this case suggests that intravitreal injection of triamcinolone in high risk eyes will also be an effective prophylactic treatment.

### EXAMPLE 2

### Use of Intravitreal Steroid Treatment for the Prevention of Neovascularisation in a Patient at High Risk

This case illustrates how intravitreal steroid treatment might be used to prevent CNV in a high risk eye.

A 67 year old patient presents with recent loss of vision in his right eye. Retinal haemorrhages and exudation are seen in the right macula. Fluorescein angiography is performed which reveals a large choroidal neovascular membrane beneath the right central macula (fovea). This is treated on its merits, but the patient is already legally blind (visual acuity less than 6/60) and likely to remain so.

Examination of the left eye, in which the visual acuity is relatively normal at 6/9, shows many large soft drusen in the central macula, some of which are greater than 500 μm in diameter, associated with coarse pigment clumping and reticular pseudodrusen in the temporal macula. The history reveals that the patient has been taking antihypertensive medication for twenty five years. The patient is otherwise healthy and looks as if he might well live another ten or twenty years.

It is explained to the patient that it has been well shown that the risk of developing neovascularisation in his left eye is roughly 90% over the next five years. Should this occur, the chance of saving reading and driving vision with conventional treatments would be less than 25%. The patient is informed that, since the formation of new blood vessels appears in this disease to be linked to chronic, low grade inflammation inside the eye, we believe that an injection of steroid into the eye may reduce the risk of developing neovascularisation in his left eye. No other preventative measures have been shown to be effective. Serious side effects of the injection are rare. The patient returns in one week, having considered his options, and elects to receive the treatment.

The patient's left eye is anaesthetised and sterilised with topical medications. An injection into the vitreous of 4 mg of triamcinolone (0.1 mL of a 40 mg/mL solution) is performed. The patient is reviewed at 1 and 6 weeks after the injection, then at 3, 6 and 12 months. After 12 months it is apparent that no complications of the procedure have ensued and the patient has maintained visual acuity of 6/9 without evidence clinically or angiographically of neovascularisation. A second injection of triamcinolone is instilled, with the patients consent, and he is reviewed with the same frequency as after the first injection. Two years after the first injection the patient's visual acuity remains 6/9. Further treatments are deferred and the patient is reviewed every 6 months.

### EXAMPLE 3

### Clinical Observations of Side Effects

Intravitreal triamcinolone presents a manageable side effect profile. Of the several hundred patients treated through the Sydney Eye Hospital over the last three years, no case of endophthalmitis, retinal detachment or vitreous haemorrhage has been reported. The commonest side effect is a modest elevation of the intraocular pressure of around 5 mmHg. This has been controlled with glaucoma medication where necessary, although if the optic nerve is not compromised and the pressure is less than 25 mmHg it is often reasonable to observe without treatment. The pressure invariably returns to normal after the drug wears off, which is usually after approximately 6 months. It is conceivable that patients will eventually develop cataract in the treated eye, but this has not been a problem with follow-up to 18 months.

The above describes some embodiments of the present invention. Modifications obvious to those skilled in the art can be made thereto without departing from the scope of this invention.

### INDUSTRIAL APPLICABILITY

It should be clear that the present invention will find wide applicability in the medical profession.

1. A method for the prevention of choroidal neovascularisation in macular degeneration in a patient requiring said prevention, comprising introducing into the vitreous of said patient an effective amount of an anti-inflammatory steroid or an ophthalmologically acceptable composition or formulation containing said anti-inflammatory steroid wherein said patient does not have choroidal neovascularisation in the eye to be treated but has an increased risk factor of developing choroidal neovascularisation.

2. The method according to claim 1 wherein the increased risk factor is based on the following criteria:
there is no evidence of choroidal neovascularisation in the eye in need of treatment but there is choroidal neovascularisation in the fellow eye; and
the patient has a further risk factor for choroidal neovascularisation.

3. The method according to claim 1 wherein the increased risk factor is manifested by early retinal pigment epithelium changes.

4. The method according to claim 3 wherein the increased risk factor is one or more of the following: soft drusen, pigment clumps or “pseudodrusen” in either eye.

5. The method according to claim 2 wherein the further risk factor is one or more of the following: ≧5 drusen which are larger than 65 μm in diameter; focal hyperpigmentation; ≧1 large drusen and systemic hypertension.

6. The method according to claim 1 wherein additional criteria which may be applicable to the patient in (a) or (b) comprise one or more of the following additional risk factors: the patient either has a family history of choroidal neovascularisation or is genetically predisposed to it; there is evidence that the patient has an immune response directed against the retina; and the patient is about to undergo intraocular surgery.

7. The method according to claim 1 wherein choroidal neovascularisation include occult and classic neovascularisation.

8. An anti-inflammatory steroid or an ophthalmologically acceptable composition or formulation containing said anti-inflammatory steroid, when used in the prevention of choroidal neovascularisation in macular degeneration, said prevention as defined in claim 1.

9. An anti-inflammatory steroid or an ophthalmologically acceptable composition or formulation containing said anti-inflammatory steroid, for use in the prevention of choroidal neovascularisation in macular degeneration, said prevention as defined in claim 1.

10. The use of an anti-inflammatory steroid or an ophthalmologically acceptable composition or formulation containing said anti-inflammatory steroid, for the manufacture of a medicament for the prevention of choroidal neovascularisation in macular degeneration, said prevention as defined in claim 1.

11. The method, steroid, composition or formulation, or use according to claim 1, wherein said steroid is in crystalline form.

12. The method, steroid, composition or formulation, or use according to claim 1, wherein the steroid is sparingly soluble in the vitreous of the eye.

13. The method, steroid, composition or formulation, or use according to claim 1, wherein the steroid is an 11-substituted 16α,17α-substituted methylenedioxy steroid of the formula: 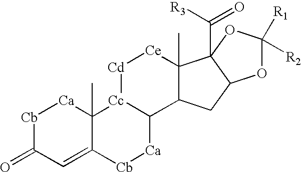
wherein 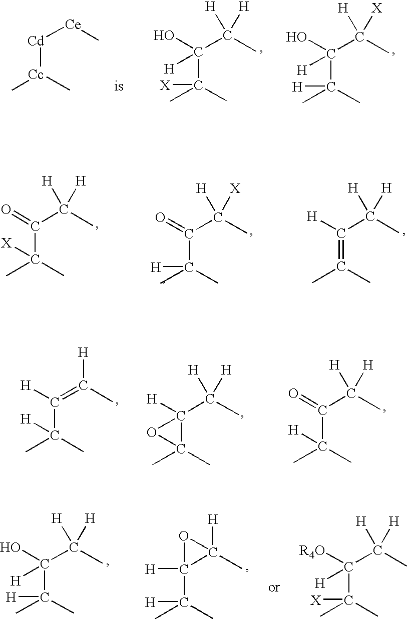
; R₁ and R₂ are hydrogen or alkyl; -Cₐ-C_{b} is —CH₂—CH₂—, —CH═CH—, 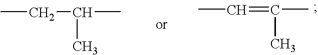
R₃ is methyl, hydroxymethyl or alkylcarbonyloxymethyl, methylaminoalkylenecarbonyloxymethyl, or phenylaminoalkylenecarbonyloxymentyl; R₄ is alkanoyl; and X is halogen.

14. The method, steroid, composition or formulation, or use according to claim 13, wherein the steroid is 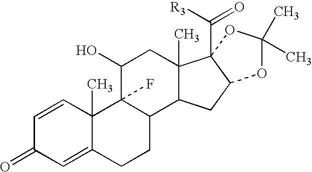
wherein R₃ is hydroxymethyl, phenylcarbonylaminoisopropylcarbonyloxymethyl, or 2,2dimethylpropylcarbonyloxymethyl.

15. The method, steroid, composition or formulation, or use according to claim 14, wherein the steroid is 9-fluoro-11,21-dihydroxy-16,17-[1-(methylethylidinebis)(oxy)]pregna-1,4-diene, 3,20-dione:

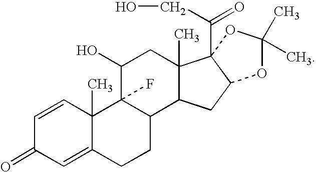

16. The method, steroid, composition or formulation, or use according to claim 15, wherein the dosage of steroid is between about 1 and about 8 mg.

17. The method, steroid, composition or formulation, or use according to claim 16, wherein the dosage is about 4 mg.

18. The method, steroid, composition or formulation, or use according to claim 13 wherein the steroid is 6,9-difluoro-11,21-dihydroxy-16,17-[(1-methylethyiidene)bis(oxy)]pregna-1,4-diene-3,20dione:

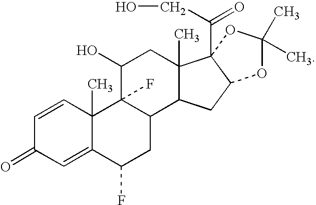

19. The method, steroid, composition or formulation, or use according to claim 18, wherein the dosage of steroid is between about 1 mg and about 8 mg.

20. The method, steroid, composition or formulation, or use according to claim 19, wherein the dosage is about 4 mg.

21. The method, steroid, composition or formulation, or use according to claim 1, wherein the macular degeneration is early onset macular degeneration, atrophic macular degeneration or neovascular macular degeneration.

22. The method, steroid, composition or formulation, or use according to claim 1, in conjunction with a further active substance.

23. The method, steroid, composition or formulation or use according to claim 22, wherein the further active substance is an anti-angiogenesis agent.

24. The method, steroid, composition or formulation, or use according to claim 23, wherein the anti-angiogenesis agent is thalidomide.

25. The method, steroid, composition or formulation, or use according to claim 24, wherein the further active substance is an antibiotic.

26. The method, steroid, composition or formulation, or use according to claim 1, in conjunction with another therapy.

27. The method, steroid, composition or formulation, or use according to claim 26, wherein the other therapy is laser treatment of the retina and the anti-inflammatory steroid is injected before or after laser treatment.

28. The method according to claim 1, wherein introduction is effected by injection; iontophoresis; through an indwelling catheter or similar device such as a tube or an injection port; or through a surgical incision.

29. The method according to claim 28 wherein the steroid is introduced in a non-erodible device; a biodegradable preparation; biodegradable micro-and nano-particles; liposomes; a drug-drug conjugate or a polymer-drug conjugate.
